(19) United States
(12) Patent Application Publication
(10) Pub. No.: US 2024/0066310 A1
(43) Pub. Date: Feb. 29, 2024

(54) GARMENT CHANGE AND WASH DETECTION IN A WEARABLE CARDIOVERTER DEFIBRILLATOR (WCD)

(71) Applicant: West Affum Holdings Corp., Grand Cayman (KY)

(72) Inventor: Robert R. Buchanan, Bothell, WA (US)

(73) Assignee: West Affum Holdings Corp., Grand Cayman (KY)

(21) Appl. No.: 17/900,772

(22) Filed: Aug. 31, 2022

(51) Int. Cl. A61N 1/39; A47G 25/14; A61B 5/316; A61N 1/04

(52) U.S. Cl. CPC A61N 1/3925; A47G 25/1407; A61B 5/316; A61N 1/0484; A61B 2562/0219; A61B 2562/0271

(57) ABSTRACT

A patient monitoring system comprises a support structure comprising a garment configured to be worn by a patient, a plurality of patient parameter electrodes configured to contact the patient's skin when the patient is wearing the garment, a patient monitoring device configured to receive one or more patient parameters via the plurality of patient parameter electrodes when the patient is wearing the garment, and a processor. The processor is configured to determine a likelihood whether the garment has been changed or washed after a predetermined period of use by the patient, and provide an indication regarding garment change or wash status based on the likelihood that the garment has been changed or washed.

### BACKGROUND

Wearable cardioverter defibrillators (WCDs) analyze patient signals to determine if the patient is experiencing a cardiac arrest. If a ventricular tachycardia/ventricular fibrillation (VT/VF) arrest is suspected, the WCD will alarm to warn the patient and bystanders of an impending shock, and if no stop signal is received then the WCD will apply one or more therapeutic shocks to the patient.

Since patients wear WCD systems and the related garments for extended periods of time, skin problems and irritations can become a concern and can limit patient wear and usage compliance. Current WCD systems have a large occurrence of skin irritation complaints, for example approximately 1400 MDR responses a year for skin inflammation or irritation. Infrequent changing or washing of the WCD garments can create new irritations or exacerbate existing skin problems. When the patient reduces wear and usage compliance due to skin inflammation or irritation, the clinician benefit of the WCD can be limited or reduced.

### DESCRIPTION OF THE DRAWING FIGURES

Claimed subject matter is particularly pointed out and distinctly claimed in the concluding portion of the specification. However, such subject matter can be understood by reference to the following detailed description when read with the accompanying drawings in which:

FIG. 1A is a diagram of a WCD system including an external defibrillator in accordance with one or more embodiments.

FIG. 1B is a diagram of a patient worn garment of the WCD system of FIG. 1A.

FIG. 2 is a diagram of components of an external defibrillator in accordance with one or more embodiments.

FIG. 3 is a diagram of a WCD remote patient data platform configured to monitor and detect whether a garment has been changed or washed in accordance with one or more embodiments.

FIG. 4 is a diagram of patient WCD system usage trends monitored by a WCD remote patient data platform in accordance with one or more embodiments.

FIG. 5 is a diagram of a garment near field communication (NFC) tag and an NFC tag reader in accordance with one or more embodiments.

FIG. 6 is a flow diagram of a method to detect a likelihood that a garment of a WCD system has not been washed or changed in accordance with one or more embodiments.

It will be appreciated that for simplicity and/or clarity of illustration, elements illustrated in the figures have not necessarily been drawn to scale. For example, the dimensions of some of the elements can be exaggerated relative to other elements for clarity. Further, if considered appropriate, reference numerals have been repeated among the figures to indicate corresponding and/or analogous elements.

### DETAILED DESCRIPTION

In the following detailed description, numerous specific details are set forth to provide a thorough understanding of claimed subject matter. It will, however, be understood by those skilled in the art that claimed subject matter can be practiced without these specific details. In other instances, well-known methods, procedures, components and/or circuits have not been described in detail.

In the following description and/or claims, the terms coupled and/or connected, along with their derivatives, can be used. In particular embodiments, connected can be used to indicate that two or more elements are in direct physical and/or electrical contact with each other. Coupled can mean that two or more elements are in direct physical and/or electrical contact. Coupled, however, can also mean that two or more elements are not in direct contact with each other, but yet can still cooperate and/or interact with each other. For example, “coupled” can mean that two or more elements do not contact each other but are indirectly joined together via another element or intermediate elements. Finally, the terms “on,” “overlying,” and “over” can be used in the following description and claims. “On,” “overlying,” and “over” can be used to indicate that two or more elements are in direct physical contact with each other. It should be noted, however, that “over” can also mean that two or more elements are not in direct contact with each other. For example, “over” can mean that one element is above another element but not contact each other and can have another element or elements in between the two elements. Furthermore, the term “and/or” can mean “and”, it can mean “or”, it can mean “exclusive-or”, it can mean “one”, it can mean “some, but not all”, it can mean “neither”, and/or it can mean “both”, although the scope of claimed subject matter is not limited in this respect. In the following description and/or claims, the terms “comprise” and “include,” along with their derivatives, can be used and are intended as synonyms for each other.

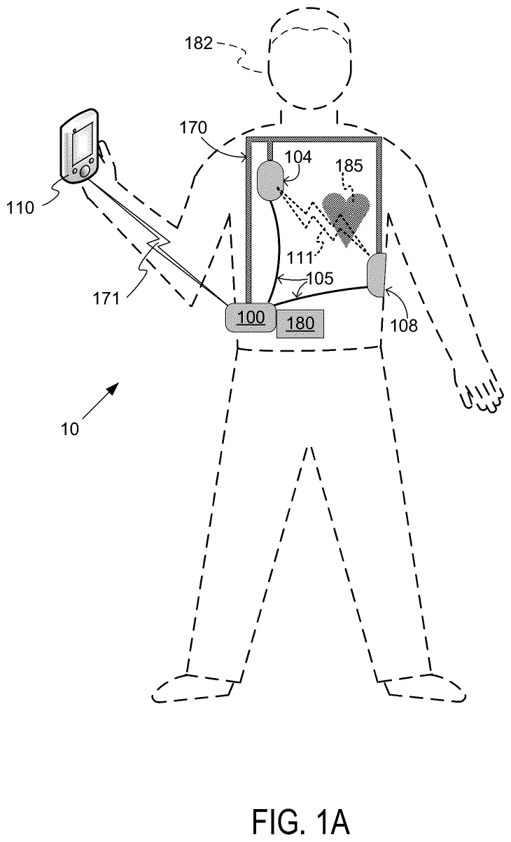

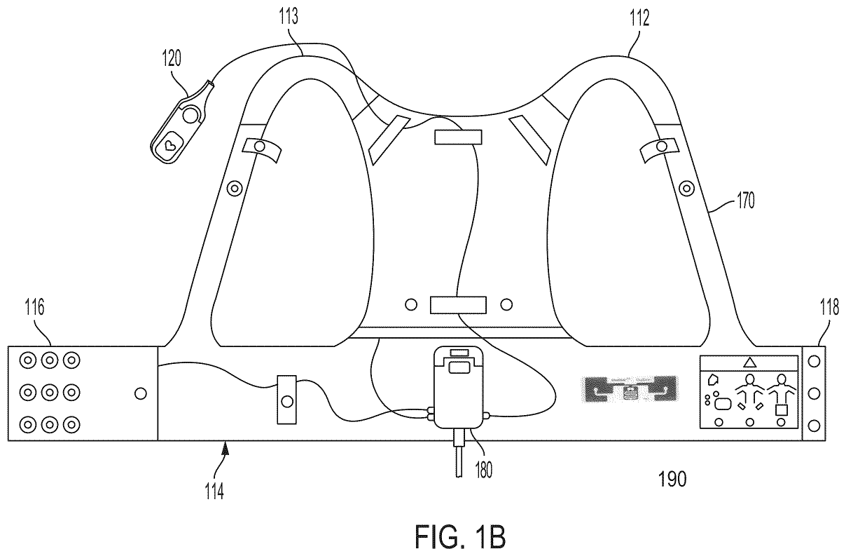

Referring now to FIG. 1A, a diagram of a WCD system including an external defibrillator in accordance with one or more embodiments will be discussed. FIG. 1A depicts components of a WCD system 10 made according to embodiments, as it might be worn by a person 182. A person such as person 182 can also be referred to as patient 182, or wearer 182 since he or she wears the WCD system 10.

The components of the WCD system of FIG. 1A include a generic support structure 170 shown relative to the body of patient 182, and thus also relative to his or her heart 185. Structure 170 could be a harness, a vest, a half-vest, one or more belts, or a garment, and so on, as per the above. Structure 170 could be implemented in a single component, or multiple components, and so on. Structure 170 is wearable by patient 182, but the manner of wearing it is not depicted, as structure 170 is depicted only generically in FIG. 1A. Structure 170 can be designed to be worn under the clothes of patient 182 and can be shaped and sized to effectively remain hidden. This can be accomplished by thin materials, design principles that avoid often-exposed areas of a patient's anatomy, such as the neck, upper chest or lower arms, and/or providing an extensive range of sizes and/or adjustability.

A wearable cardiac defibrillator (WCD) system 10 can be configured to defibrillate a patient 182 who is wearing it by delivering electrical charge to the patient's body in the form of an electric shock delivered in one or more pulses. The components of the WCD system 10 of FIG. 1A include a sample external defibrillator 100 made according to embodiments, and sample defibrillation electrodes 104 and 108 which are coupled to external defibrillator 100 via electrode leads 105. Defibrillator 100 and defibrillation electrodes 104 and 108 are coupled to support structure 170. As such, many of the individual components of defibrillator 100 can be therefore coupled to support structure 170. When defibrillation electrodes 104 and 108 make good electrical contact with the body of patient 182, defibrillator 100 can administer, via electrodes 104 and 108, a brief, strong electric pulse 111 through the body. In some examples, defibrillation electrodes can be configured to be in electrical contact with the patient's skin when the patient is wearing the garment or support structure 170 and/or when defibrillator 100 is delivering therapy to the patient. In some examples, defibrillation electrodes 104 and 108 can be in electrical contact with the patient's skin via an intermediate element such as a silver mesh between the defibrillation electrodes 104 and 108 and the patient's skin. Pulse 111, also known as a defibrillation shock or therapy shock, is intended to go through and restart heart 185 in an effort to save the life of patient 182. Pulse 111 can further include one or more pacing pulses, and so on. A defibrillator typically decides whether to defibrillate or not based on an electrocardiogram (“ECG”) signal of the patient. Defibrillator 100, however, can defibrillate or not defibrillate also based on other inputs.

In the example of FIG. 1A, defibrillator 100 includes additional individual components, as will be described in more detail later in this document. Briefly, these additional components include a power source or energy storage module that is configured to store an electrical charge, a discharge circuit, and one or more processors. In this example, the components also include a communication module that is integrated with the defibrillation unit in a single electronics module, although the communication module can be provided in an electronics module of the WCD system 10 separately from that of the shown defibrillator 100.

In the example of FIG. 1A, defibrillator 100 is shown in the front of the patient. In some embodiments, one or more components of a WCD system 10 are preferably ergonomically designed to fit the lumbar region of the body. The lumbar region is sometimes referred to as the lower spine or as an area of the back in its proximity. A component such as an electronics module can be discreetly worn on the body under a patient's clothes when placed in a lumbar pack/carrying case or carried in a common accessory such as a purse or backpack, effectively hiding it in plain sight. Such an electronics module can include one or more components of the WCD system 10.

The components of the WCD system 10 of FIG. 1A also include an action unit 180. Action unit 180 can also be referred to as a hub or an outside monitoring device. Action unit 180 can be a device for patient 182 to exchange information with the WCD system 10. In particular, action unit 180 can have a user interface that is configured to enable patient 182 to read system messages and enter action inputs.

Action unit 180 can be configured to be coupled to support structure 170. In some embodiments, action unit 180 is integrated with the one or more processors in a single electronics module, for example the same electronics module that includes defibrillator 100. In some embodiments, action unit 180 is electrically coupled with the module of defibrillator 100 via a cable, which can be a permanent cable or a Universal Serial Bus (USB), Firewire connection, or similar cable or connector.

For use, patient 182 can reach into their clothes to access action unit 180. In embodiments where a cable is used, patient 182 can bring action unit 180 to a comfortable position for reading the system messages and entering the action inputs. Accordingly, patient 182 can access and control various functions of the WCD system via action unit 180.

According to embodiments, some of the WCD system functions that can be controlled by action unit 180 can instead be controlled by a mobile communication device 110, redundantly or not. In such embodiments, patient 182 carries mobile communication device 110 on their person for typically much of the day. Patient 182 can carry device 110 in a pocket, in a special holder, or even wear it on their wrist. Patient 182 can use device 110 to communicate with the WCD system 10, which is why patient 182 can also be referred to as user 182. Mobile communication device 110 has a user interface that is configured to enable patient 182 to enter inputs that in this document are often called wireless inputs. Wireless communication links can be established and used in embodiments, for exchanging data, voice, and so on. A wireless communication link is also sometimes referred to as “commlink”.

In some examples, a mobile communication device such as device 110 can be a custom-made device that is part of the WCD system 10. If made to look substantially like a common, commercially available mobile communication device, it might help preserve the privacy of patient 182 as to the fact that he or she is wearing a medical device, and thus also help preserve their dignity.

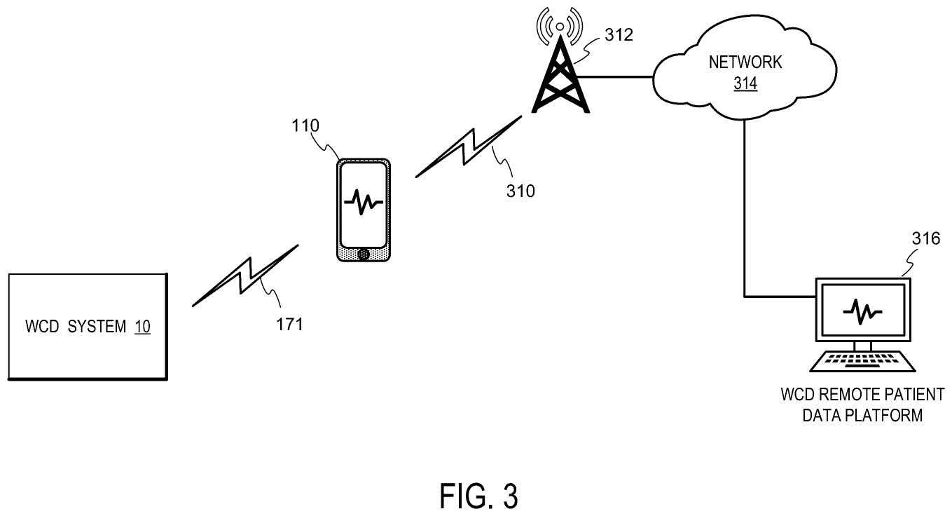

Alternately, a mobile communication device such as device 110 can be a wireless telephone, a smartphone, a Personal Digital Assistant (PDA), a personal electronic device, a pager, a laptop computer, a tablet, an e-reader, and so on. It can have an application, or “app”, made according to embodiments, so as to perform various functions as described. In such embodiments, mobile communication device 110 can communicate with a wireless service provider network via a remote commlink as shown in FIG. 3 and described with respect to FIG. 3 below. In some examples, a “remote commlink” can refer to a wireless communication link established between devices that are at least 500 feet (150 m) away from each other, and typically farther, such as a cellular communication link. In such instances, the remote commlink can be used for a number of other functions, such as dialing an emergency number (e.g., 911 in the US), which can also be accessible via the mobile communication device 110 directly. In addition, the location of the patient 182 can be determined using Global Positioning System (GPS) for example using appropriate hardware in mobile communication device 110, action unit 180, and/or defibrillator 100. If the WCD system 10 and the mobile communication device 110 have been paired and one of them knows that it is physically close to the other, GPS information can become known and communicated to Emergency Medical Services (EMS) services. The mobile communication device 110 can provide a redundant communication path for the data of the WCD system 10. This redundant communication path might be used as a secondary communication path for remote monitoring data if a primary, in-house internet path is not available for the WCD system 10 to report. The remote commlink can also be used by a remote caregiver to provide patient 182 with troubleshooting assistance, motivational feedback, and so on.

Mobile communication device 110 can thus be configured to establish a local commlink 171 with a communication module of the WCD system 10, which can be inside the same module as defibrillator 100 and/or in action unit 180. If mobile communication device 110 is a wireless telephone or other independent standalone communication device, a local commlink can be established first pursuant to some authentication. Local commlink 171 can be established by the initiative of mobile communication device 110, the communication module, or both. For purposes of this document, a “local commlink” can refer to a wireless communication link established between devices that are at most 50 feet (15 m) away from each other, and typically closer, such as when patient 182 is holding device 110. Local commlink 171 can be a wireless link. Data can be exchanged via local commlink 171, in either direction, or in both directions. In embodiments, local commlink 171 uses radio transmission technology that can be broadband and/or shortwave. Local commlink 171 can use Bluetooth technology, Wi-Fi technology, Zigbee, or other suitable short-range wireless technology.

Referring now to FIG. 1B, a diagram of a patient worn garment of the WCD system of FIG. 1A will be discussed. The garment shown in FIG. 1B comprises one example of the support structure 170 of FIG. 1A shown in a vest configuration. The garment 170 can include shoulder straps 112 and 113 to be placed over the shoulders of the patient 182 and for support of the support garment 170. The garment 170 can include a belt portion 114 to be fastened around the waist of the patient 182. The belt portion 114 can include various fasteners 116 and 118, for example closure snaps, to allow the garment 170 to be fitted to different sized users. Action unit or hub 180 can be attached to the back side of the garment 170, for example on or near the belt portion 114, to allow various cables to be connected to the action unit 180 including alert button (or divert button) 120 and cabling to connect to the therapy/defibrillator electrodes and ECG electrodes. Action unit or hub 180 can couple with an appropriate receptable of the garment 170 for ease of attachment and detachment, for example when the garment 170 is to be changed or washed. In some embodiments, support structure 170 can comprise a vest-like fabric garment to be worn on the patient's body underneath an outer shirt or other clothing to allow the electrodes to contact the patient's skin and hold the electrodes in close proximity to and/or direct contact with the patient's skin. Such an arrangement allows for the WCD 10 to obtain ECG signals from the patient 182 and to allow one or more shocks to be applied to the patient 182 when appropriate.

In some examples, garment 170 can include an identifier (ID) tag 190 that can be used to uniquely identify the particular garment 170 being used or worn by the patient 182. In some examples as will be discussed further below, ID tag 190 can comprise a radio-frequency ID (RFID) tag with an identifier that can be read by an appropriate RFID reader, for example an RFID reader disposed in action unit or hub 180. In some further examples, ID tag 190 can include circuitry or other mechanism that is configured to determine whether garment 170 has been exposed to water, for example when the garment 170 is washed by the patient 182 or other user. It should be noted that the ID tag 190 can be disposed at any various location on the garment 170, and the scope of the disclosed subject matter is not limited in this respect.

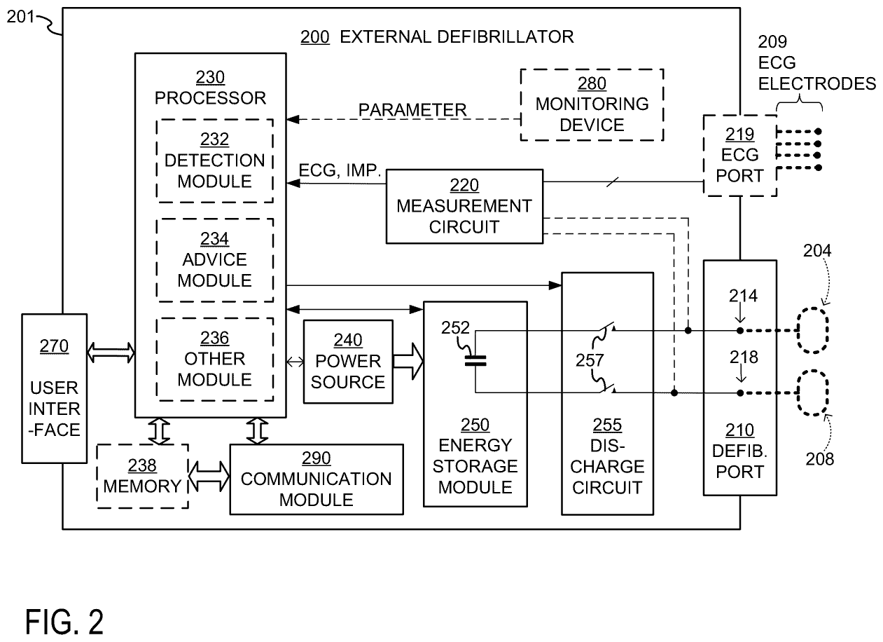

Referring now to FIG. 2, a diagram of components of an external defibrillator in accordance with one or more embodiments will be discussed. FIG. 2 is a diagram showing individual components of an external defibrillator 200, which is made according to embodiments, and which can correspond to defibrillator 100 of FIG. 1A. These individual components can be, for example, those included in the module that includes defibrillator 100 of FIG. 1A. The components shown in FIG. 2 can be provided in a housing 201, which is also known as casing 201. In some examples, some of the components of defibrillator 200 can be housed in action unit or hub 180. In general, defibrillator 200 can embody at least some or all of the components of WCD system 10 and as such defibrillator 200 and WCD system 10 can be used interchangeably.

Defibrillator 200 is intended for a patient 182 who would be wearing the WCD system 10, such as patient 182 of FIG. 1A. Defibrillator 200 can further include a user interface 270, which can be the same as action unit 180. User interface 270 can thus be used by patient 182, or a bystander at a scene where the patient can experience sudden cardiac arrest (SCA). The bystander can be a person familiar with patient 182, a stranger, a trained person, etc. In some scenarios the bystander can be a rescuer, and so on.

User interface 270 can be made in a number of ways. User interface 270 can include output devices, which can be visual, audible, or tactile, for communicating to a user. For example, an output device can be a light, or a screen to display what is detected and measured and provide visual feedback to a rescuer for their resuscitation attempts, and so on. Another output device can be a speaker, which can be configured to issue voice prompts, etc. Sounds, images, vibrations, and anything that can be perceived by a user can also be called human perceptible indications. User interface 270 can also include input devices for receiving inputs from users. Such input devices can additionally include various controls, such as pushbuttons, keyboards, touchscreens, a microphone, and so on. An input device can be a cancel switch, which is sometimes called a “live-man” switch, an “I am OK” switch, a “divert therapy” switch, etc., which in some examples can correspond to alert button 120 of FIG. 1B. In some embodiments, actuating the cancel switch can prevent the impending delivery of a shock.

Defibrillator 200 can optionally include a monitoring device 280. Device 280 can be configured to monitor at least one local parameter. A local parameter can be a physiological parameter of patient 182, or a parameter of the WCD system 10, or a parameter of the environment, as will be described later in this document. Patient physiological parameters include, for example, those physiological parameters that can be of any help in detecting by the wearable defibrillation system whether the patient needs a shock, plus optionally their medical history and/or event history. Examples of such parameters include the patient's ECG, blood oxygen level, blood flow, blood pressure, blood perfusion, pulsatile change in light transmission or reflection properties of perfused tissue, heart sounds, heart wall motion, breathing sounds and pulse. Accordingly, the monitoring device could include a perfusion sensor, a pulse oximeter, a Doppler device for detecting blood flow, a cuff for detecting blood pressure, an optical sensor, illumination detectors and perhaps sources for detecting color change in tissue, a motion sensor, a device that can detect heart wall movement, a sound sensor, a device with a microphone, an SpO2 sensor, and so on. Pulse detection is taught at least in U.S. Pat. No. 8,135,462, which is hereby incorporated by reference in its entirety. In addition, a person skilled in the art can implement other ways of performing pulse detection.

Patient state parameters include recorded aspects of patient 182, such as motion, posture, whether they have spoken recently plus also what they said, and so on, plus optionally the history of these parameters. Alternatively, one of these monitoring devices could include a location sensor such as a Global Positioning System (GPS) location sensor. Such a sensor can detect the location, plus a speed can be detected as a rate of change of location over time. Many motion detectors output a motion signal that is indicative of the motion of the detector, and thus of the patient's body. Patient state parameters can be very helpful in narrowing down the determination of whether sudden cardiac arrest (SCA) is indeed taking place.

Defibrillator 200 typically includes a defibrillation port 210, such as a socket in housing 201. Defibrillation port 210 includes electrical nodes 214 and 218. Leads of defibrillation electrodes 204 and 208, such as leads 105 of FIG. 1A, can be plugged in defibrillation port 210, so as to make electrical contact with nodes 214 and 218, respectively. Electrodes 204 and 208 can be electrodes 104 and 108. Either way, defibrillation port 210 can be used for guiding, via electrodes, to the wearer the electrical charge that has been stored in energy storage module 250. The electric charge will be the shock for defibrillation, pacing, and so on.

Defibrillator 200 can optionally also have an ECG port 219 in housing 201, for plugging in sensing electrodes 209, which are also known as ECG electrodes and ECG leads. It is also possible that sensing electrodes 209 can be connected continuously to ECG port 219, instead. Sensing electrodes 209 can help sense an ECG signal, for example a 12-lead signal, or a signal from a different number of leads, especially if they make good electrical contact with the body of the patient. Sensing electrodes 209 can be attached to the inside of support structure 170 for making good electrical contact with the patient, similarly as defibrillation electrodes 204 and 208. In some examples, defibrillation electrodes 204 and 208 can be configured to be in electrical contact with the patient's skin via an intermediate element such as a silver mesh, for example when the patient is wearing the garment or support structure 170 and/or when defibrillator 200 is delivering therapy to the patient.

Defibrillator 200 also includes a measurement circuit 220. Measurement circuit 220 receives physiological signals of the patient from ECG port 219, if provided. Even if defibrillator 200 lacks ECG port 219, measurement circuit 220 can obtain physiological signals through nodes 214 and 218 instead, when defibrillation electrodes 204 and 208 are attached to the patient 182. In these cases, the patient's ECG signal can be sensed as a voltage difference between electrodes 204 and 208. In addition, impedance between electrodes 204 and 208 and/or the connections of ECG port 219 can be sensed. Sensing the impedance can be useful for detecting, among other things, whether these electrodes 204 and 208 and/or sensing electrodes 209 are not making good electrical contact with the patient's body. These patient physiological signals can be sensed, when available. Measurement circuit 220 can then render or generate information about them as physiological inputs, data, other signals, and so on. More strictly speaking, the information rendered by measurement circuit 220 is output from it, but this information can be called an input because it is received by a subsequent device or functionality as an input.

A WCD system 10 according to embodiments also includes one or more processors, of which defibrillator 200 shows only one processor 230. The one or more processors can be implemented in any number of ways. Such ways include, by way of example and not of limitation, digital and/or analog processors such as microprocessors and Digital Signal Processors (DSPs), controllers such as microcontrollers, software running in a machine, programmable circuits such as Field Programmable Gate Arrays (FPGAs), Field-Programmable Analog Arrays (FPAAs), Programmable Logic Devices (PLDs), Application Specific Integrated Circuits (ASICs), any combination of one or more of these, and so on.

The one or more processors can be configured to receive the action inputs that have been entered via the user interface of action unit 180, and the wireless inputs that have been entered via the user interface of mobile communication device 110. In addition, the one or more processors can be configured to perform various functions, for example by causing various components to operate in certain ways. In some embodiments, the performance of these functions can have aspects that are controlled by any received action inputs and wireless inputs.

A number of functions are possible according to embodiments, which the one or more processors can be configured to perform. Some of these functions are described later in this document. Of these functions, a first function can be to cause the patient to be defibrillated. This first function can be performed by controlling discharge circuit 255 to discharge the electrical charge stored in power source 240 through patient 182, while patient 182 is wearing support structure 170.

In the example of FIG. 2, for this first function processor 230 can be considered to have a number of modules. One such module can be a detection module 232. Detection module 232 can include a ventricular fibrillation (“VF”) detector. The patient's sensed ECG from measurement circuit 220, which can be available as physiological inputs, data, or other signals, can be used by the VF detector to determine whether the patient is experiencing VF. Detecting VF is useful because VF results in SCA. Detection module 232 can also include a ventricular tachycardia (“VT”) detector, and so on.

Another such module in processor 230 can be an advice module 234, which generates advice for what to do. The advice can be based on outputs of detection module 232. There can be many types of advice according to embodiments. In some embodiments, the advice is a shock/no shock determination that processor 230 can make, for example via advice module 234. The shock/no shock determination can be made by executing a stored Shock Advisory Algorithm. A Shock Advisory Algorithm can make a shock/no shock determination from one or more of ECG signals that are captured according to embodiments and determining whether a shock criterion is met. The determination can be made from a rhythm analysis of the captured ECG signal or otherwise.

In some embodiments, when the decision is to shock, an electrical charge is delivered to the patient. Delivering the electrical charge is also known as discharging. Shocking can be for defibrillation, pacing, and so on.

Processor 230 can include additional modules, such as other module 236, for other functions. In addition, if internal monitoring device 280 is indeed provided, it can be operated in part by processor 230, and so on. Defibrillator 200 optionally further includes a memory 238, which can work together with processor 230. Memory 238 can be implemented in any number of ways. Such ways include, by way of example and not of limitation, volatile memories, nonvolatile memories (NVM), read-only memories (ROM), random access memories (RAM), magnetic disk storage media, optical storage media, smart cards, flash memory devices, any combination of these, and so on. Memory 238 is thus a non-transitory storage medium. Memory 238, if provided, can include programs for processor 230, which processor 230 can be able to read and execute. More particularly, the programs can include sets of instructions in the form of code, which processor 230 can be able to execute upon reading. Executing is performed by physical manipulations of physical quantities, and can result in the functions, processes, actions and/or methods to be performed, and/or the processor to cause other devices or components or blocks to perform such functions, processes, actions and/or methods. The programs can be operational for the inherent needs of processor 230 and can also include protocols and ways that decisions can be made by advice module 234. In addition, memory 238 can store prompts for the user of user interface 270 if this user is a local rescuer. Moreover, memory 238 can store data. The data can include patient data, system data and environmental data, for example as learned by internal monitoring device 280 and outside monitoring device 180. The data can be stored in memory 238 before it is transmitted out of defibrillator 200 or stored there after it is received by defibrillator 200.

As mentioned previously, defibrillator 200 also includes a power source 240. To enable portability of defibrillator 200, power source 240 typically includes a battery. Such a battery is typically implemented as a battery pack, which can be rechargeable or not. Sometimes a combination is used of rechargeable and non-rechargeable battery packs. Other embodiments of power source 240 can include an AC power override, for where AC power will be available, an energy storage capacitor, and so on. In some embodiments, power source 240 is controlled by processor 230.

Defibrillator 200 additionally includes an energy storage module 250, which can thus be coupled to the support structure of the WCD system 10. Module 250 is where some electrical energy is stored in the form of an electrical charge, when preparing it for sudden discharge to administer a shock. Module 250 can be charged from power source 240 to the right amount of energy, as controlled by processor 230. In typical implementations, module 250 includes a capacitor 252, which can be a single capacitor or a system of capacitors, and so on. As described above, capacitor 252 can store the energy in the form of electrical charge, for delivering to the patient.

As mentioned previously, defibrillator 200 moreover includes a discharge circuit 255. When the decision is to shock, processor 230 can be configured to control discharge circuit 255 to discharge through the patient the electrical charge stored in energy storage module 250. When so controlled, circuit 255 can permit the energy stored in module 250 to be discharged to nodes 214 and 218, and from there also to defibrillation electrodes 204 and 208. Circuit 255 can include one or more switches 257. Switches 257 can be made in a number of ways, such as by an H-bridge, and so on. Circuit 255 can also be controlled via user interface 270.

As mentioned previously, defibrillator 200 includes a communication module 290, for establishing one or more wired or wireless communication links with other devices of other entities, such as a remote assistance center, Emergency Medical Services (EMS), device 110, and so on. In other embodiments, the communication module need not be in the same housing 201 as defibrillator 200. Module 290 can also include an antenna, portions of a processor, and other sub-components as can be deemed necessary by a person skilled in the art. This way, data and commands can be communicated via commlinks, such as patient data, event information, therapy attempted, cardiopulmonary resuscitation (CPR) performance, system data, environmental data, and so on.

Defibrillator 200 can optionally include other components. Returning to FIG. 1A, in embodiments, one or more of the components of the shown WCD system have been customized for patient 182. This customization can include a number of aspects. For instance, support structure 170 can be fitted to the body of patient 182. For another instance, baseline physiological parameters of patient 182 can be measured, such as the heart rate of patient 182 while resting, while walking, motion detector outputs while walking, etc. Such baseline physiological parameters can be used to customize the WCD system, in order to make its diagnoses more accurate, since bodies behave differently. For example, such parameters can be stored in a memory of the WCD system, and so on. A programming interface can be made according to embodiments, which receives such measured baseline physiological parameters. Such a programming interface can input automatically in the WCD system the baseline physiological parameters, along with other data.

It will be appreciated that embodiments give patient 182 the option to control the performance of a number of functions of the WCD system via mobile communication device 110, as opposed to action unit 180. Using device 110 will attract less attention in public places where others can be watching, than using action unit 180. In addition, using device 110 will be less distracting to people familiar with patient 182, and the fact that this patient needs to be attending to their WCD system. As such, patient 182 will have one less deterrent from exhibiting good compliance in actually wearing their WCD system daily.

Embodiments make various allocations as to which of action unit 180 and mobile communication device 110 can affect which functions of the one or more processors of the WCD system 10. In some embodiments there is redundancy, in that one or more functions can be accessed from either action unit 180 or mobile communication device 110, meaning aspects of the performance of these functions can be controlled either by received action inputs or by received wireless inputs. In some embodiments there is exclusivity, in that one or more functions can be accessed from either action unit 180 or mobile communication device 110, but not both. In some embodiments there is redundancy for some functions and exclusivity for others.

The allocations can be made by considering the context that functions can be performed in, in terms of criticality and afforded privacy. For example, there can be a preference that some initialization functions that are operated when patient 182 is initially fitted with the WCD system 10 at the doctor's office be exclusively accessible and controllable by action unit 180, and not accessible by mobile communication device 110. For another example, there can be a preference that functions which patient 182 is expected to perform periodically be accessible from mobile communication device 110. For some of these choices it can be further considered that, in case of an emergency, action unit 180 can be more reliable if it does not need to be separately powered, or for a wireless network to be operating.

Referring now to FIG. 3, a diagram of a WCD remote patient data platform configured to monitor and detect whether a garment has been changed or washed in accordance with one or more embodiments will be discussed. As shown in FIG. 3, WCD remote patient data platform 316 can be configured to communicate with WCD system 10, either directly or through mobile communication device 110, via wireless network 312 and/or network 314. WCD system 10 can be configured to communicate with mobile communication device via commlink 171, or directly with network 312 via commlink 310. Mobile communication device 110 can be configured to communicate with network 312 via commlink 310, and in some embodiments mobile communication device 110 can be configured to operate as an intermediary between WCD system 10 and network 312. In some examples, wireless network 312 can comprise a wireless local area network (WLAN) such as a Wi-Fi network or a wireless wide area network (WWAN) such as a cellular telephone network. In any event, wireless network 312 can be coupled with WCD remote patient data platform 316 via a network 314 such as the Internet.

In some examples, WCD remote patient data platform 316 can comprise the CareStation™ remote patient data platform from Kestra Medical Technologies of Kirkland, Washington, USA. In such examples, WCD remote patient data platform 316 can be configured to provide management of WCD system 10 including one or more patient parameters of patient 182. WCD remote patient data platform 316 can be configured to monitor patient usage and wear compliance of WCD system 10, including monitoring how long the patient has been wearing garment 170 without being changed or washed. In some examples, WCD remote patient data platform 316 can monitor usage trends of WCD system 10 to identify the wear time of garment 170 and how long garment 170 has been used for an extended period without changing to a new, clean garment 170 and/or without washing the present garment 170. In other examples, each garment 170 used and worn by the patient 182 can include a unique identifier (ID) tag 190 that identifies the particular garment being worn by patient 182. WCD system 10 can include a tag reader that can read the garment ID stored in the ID tag 190 to determine which garment 170 is being worn by the patient 182 while the WCD system 10 is being used, and how long it has been since the garment 170 has been changed to a new or different garment 170 and/or washed.

In additional examples, the ID tag 190 can include circuitry or other mechanism to detect whether the garment 170 has been exposed to moisture, water, or other liquid and therefore washed by the patient 182 or another person. In some examples, the ID tag 190 can include other sensors to detect wither or not the garment 170 has been washed. For example, the ID tag 190 can include a temperature sensor to detect whether the garment has been exposed to the hot water of a clothes washing machine. The hot water temperature of a clothes washing machine is likely to be 130 degrees Fahrenheit or greater which is greater than the temperature of the patient's body which is around 98 degrees Fahrenheit. In another example, the ID tag 190 can include an accelerometer to detect the agitation or spinning applied to the garment 170 by a clothes washing machine or a clothes drier. The functionality to monitor whether and for how long the garment 170 has been changed or washed can be implemented in whole or in part by WCD system 10, in whole or in part by mobile communication device 110, or in whole or in part by WCD remote patient data platform 316. These examples will be discussed in further detail below.

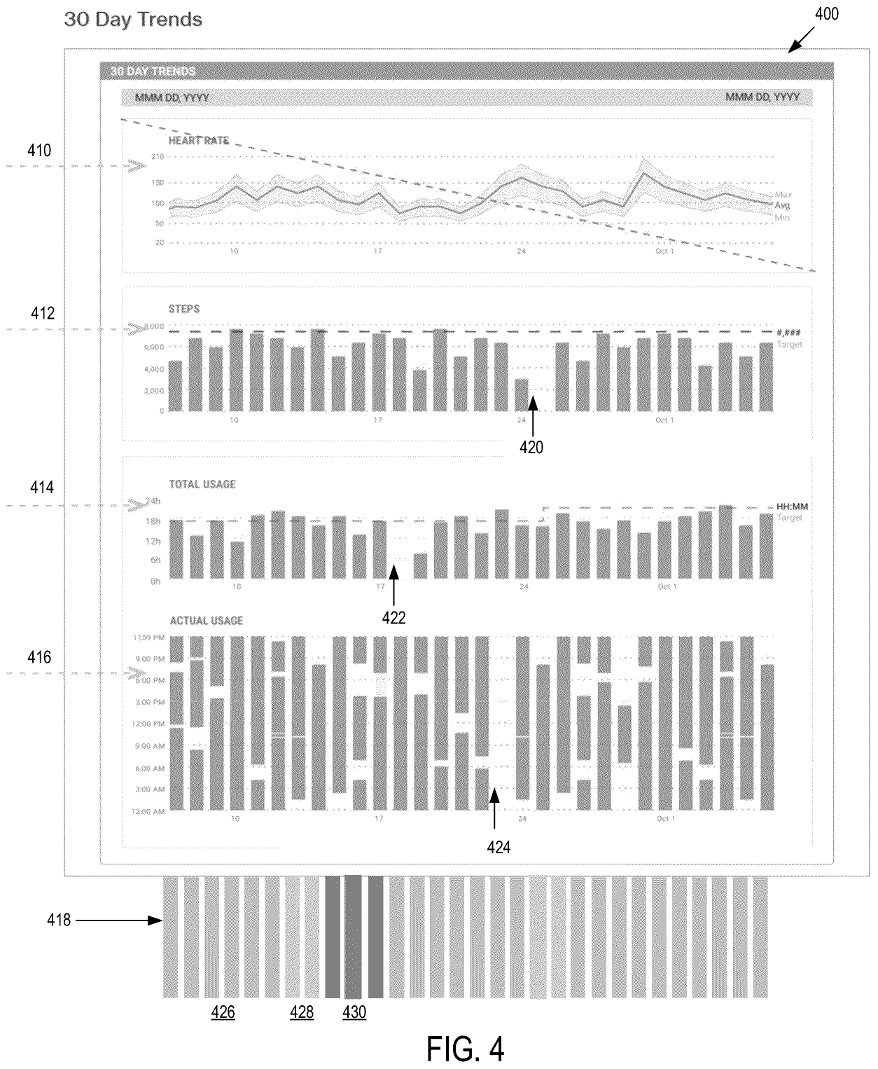

Referring now to FIG. 4, a diagram of patient WCD system usage trends monitored by a WCD remote patient data platform in accordance with one or more embodiments will be discussed. Diagram 400 illustrates a predetermined period of trends of usage of WCD system 10 by a patient 182, for example a 30-day period. These usage trends can be tracked by WCD system 10 itself, by an app running on mobile communication device 110, and/or by an application running on WCD remote patient data platform 316, by any of these devices individually or in any combination thereof. Any patient parameter measured by one device can be transmitted to any one of the other devices for example as shown in FIG. 3. Such an arrangement can allow a patient parameter obtained by one device to be logged and tracked by another device, and the scope of the disclosed subject matter is not limited in this respect.

In some examples, the usage trends can include a heart rate trend 410 tracked over the predetermined period and tracked on a graph of heart rate versus day. The tracked heart rate can include a maximum heart rate, a minimum heart rate, and/or an average heart rate logged on each tracked day. A heart rate being logged on a given day can mean that the patient 182 is using WCD system 10 and therefore wearing garment 170. If a heart rate is not logged on a given day, this can indicate that the patient 182 may not be using WCD system 10 and therefore not wearing garment. Heart rate can be measured via the ECG signal measured by WCD system 10 or can be measured by mobile communication device 110, for example where mobile communication device 110 comprises a heart rate monitor or a smart watch worn on the patient's wrist that includes heart rate monitoring functionality. Similarly, the usage trends can include a steps trend 412 wherein the number of steps taken by the patient 182 each day can be measured, for example where WCD system 10 and/or mobile communication device 110 includes a pedometer function. The vertical axis of the steps trends 412 can represent the number of steps taken on a given day represented on the horizontal scale. The vertical scale can be based on the maximum values of all of the step data and can include a target line for the patient 182 and the current target value for a given day.

The usage trends can also include a system total usage trend 414 which tracks the total hours each day that the patient 182 is using WCD system 10 and therefore wearing garment 170. Similar to the step trends 414, the system total usage trend 414 can include a usage target line and a current usage target value for a given day. The system total usage trend 414 optionally can be paired with an actual usage trend 416 which tracks the individual periods of time during a given day during which the WCD system 10 is being used by the patient 182.

In accordance with one or more embodiments, a determination can by made when a patient 182 as likely not changed or washed garment 170 based at least on part on the usage trend data tracked by any one or more of WCD system 10, mobile communication device 110, or WCD remote patient data platform 316. The purpose of tracking such usage can be to ensure that the patient 182 has not worn the same garment 170 for too long of a period without being washed or being changed to another fresh and clean garment 170 in order to reduce or prevent skin irritation issues that can arise from extended usage of the same garment 170. The number of days likely without such a garment change or wash can be represented at graph 418 which can indicate a likelihood score or category. For example, a first period 426 can indicate when a fresh garment 170 is being used. In the example shown, period 426 can represent six days of usage. Since the usage trends indicate that the WCD system 10 has been used continuously for these six days, a second period 428 can indicate that the garment 170 has been continuously used for several days without having been changed or washed, and caution can be advised, for example for a period of two days. Then, a third period of warning 430 can by indicated by the next three days meaning that the patient 182 has likely gone too long without changing or washing the garment 170 wherein a risk of skin issues may occur, and a warning can be indicated.

The usage trends of diagram 400 can be used to arrive at these above-mentioned determinations, for example based at least in part on one or more usage trends indicating which days and for how long the WCD system 10 likely has been used, and when the WCD system 10 may not be being used by the patient 182. For example, as shown at 420 of step trend 412, no steps by the patient 182 have been logged and therefore this can indicate a likelihood that the patient may not be using the WCD system 10. This likelihood can be confirmed or checked against one or more other trends such that the patient 182 may still be using WCD system 10 but may have been bedridden on that day, and no steps were taken. As shown at 422 of total usage trend 414, an absence of use of WCD system 10 can indicate that the patient 182 may be washing the garment 170 on that day and therefore the usage warning of period 430 of graph 418 can be reset to the first period 426. In some examples, when the patient 182 changes or washes garment 170, the garment wearing or washing period can be reset by the patient 182 or another person by using a user interface 270 of the WCD system 10 or a user interface of the mobile communication device 110 to reset to the first period 426.

Similarly, actual usage trend 416 can include gaps of time throughout a given day as shown at 424 which can indicate a period of time in which the garment 170 has an opportunity for being changed or washed and therefore likely has been changed or washed. For example, a gap of at least an hour or more in a given day of actual usage trend 416 may be a sufficient length of time during which garment 170 can be changed or washed, and therefore the device tracking the usage and wear trend can use this information in determining whether garment 170 has likely been changed or washed or not. Furthermore, the usage trend data shown in diagram 400 can be used in conjunction with other indicators that may be helpful in determining whether garment 170 likely has been washed or changed to another fresh garment 170. Such other indicators can include an ID tag 190 that uniquely identifies a particular garment wherein the ID of the garment 170 can be read by an appropriate device in WCD system 10 and/or mobile communication device 110. In some examples, the indicators can be an ID tag or similar device that is capable of detecting contact with moisture or water such that an indication may be made that the garment 170 has been washed. Examples of these indicators are discussed further below.

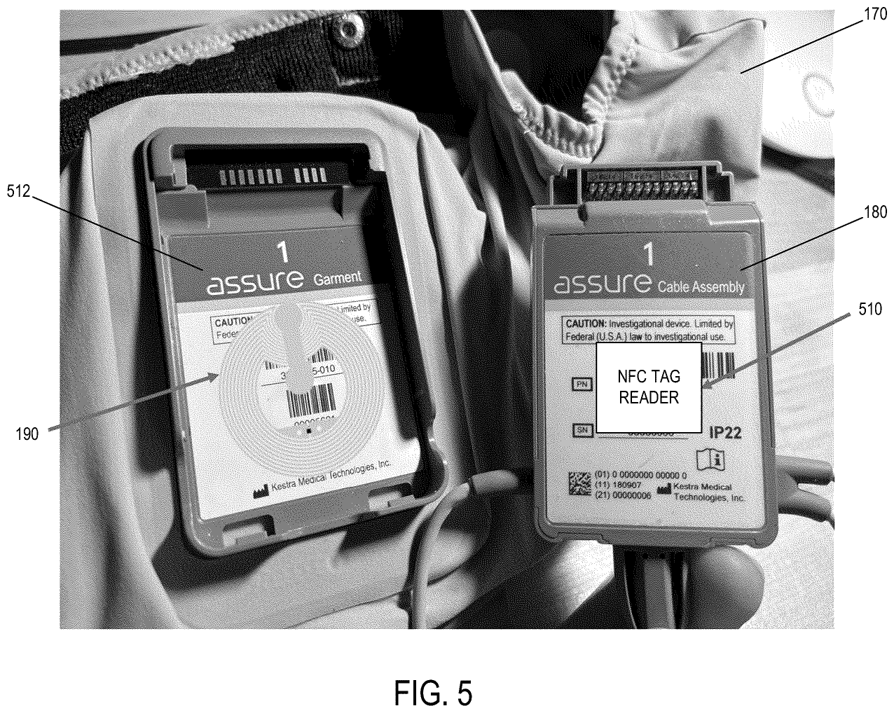

Referring now to FIG. 5, a diagram of a garment near field communication (NFC) tag and an NFC tag reader in accordance with one or more embodiments will be discussed. In the example shown in FIG. 5, garment 170 can include an identifier (ID) tag 190 that comprises a near field communication (NFC) tag on which data can be stored that is capable of being read by an appropriate NFC tag reader 510 when the NFC tag reader 510 is in sufficient close proximity to NFC tag 190. For example, the data stored by the NFC tag 190 can be a serial number specific to the garment 170 on which the NFC tag 190 is affixed. In some embodiments, NFC tag reader 510 can be included within the housing of action device or hub 180 and incorporated with the circuitry thereof. Garment 170 can include a receptacle 512 for receiving hub 180, and the NFC tag 190 can be disposed within the receptacle 512. When hub 180 is inserted into the receptacle 512 of garment 170, the NFC tag reader 510 and NFC tag 190 can be in sufficiently close proximity to one another to allow the NFC tag reader 510 to read NFC tag 190. Such an arrangement allows the NFC tag reader 510 to read the ID of the particular garment 170 so that it can be determined whether the same garment 170 is being used or whether a different garment 170 being used. In some cases, a patient 182 can have two garments 170 (or even more garments) each with a unique ID stored in its respective NFC tag 190 so that the WCD system 10 can determine which garment 170 is being used during which particular usage times, for example which garment 170 is being used on any given day. In addition, the amount of time during which the hub 180 is coupled in receptacle 512 can also be tracked, which can provide an indication of how long a garment 170 is being used on any given day and for how many consecutive days. The device tracking and logging the WCD system 10 usage and the wearing of the garment 170 can be updated for the days on which the patient 182 has changed the garment 170.

Thus, in some examples, if the patient 182 has only one garment 170, the WCD system 10 can detect when the hub 180 has been removed from the garment 170 and for how long in order to track or indicate the likelihood that the garment 170 has been washed during the removal period. For example, very short removal periods on the order of minutes may indicate that the hub 180 has not been removed from garment 170 for a long enough period of time to wash the garment 170. Longer removal periods on the order of an hour or longer can indicate that the hub 180 has been removed from garment 170 for a long enough period of time in which to wash the garment 170. In other examples, if the patient has two garments 170, the WCD system 10 can detect for how long a first garment 170 has been used without switching to a second garment 170, and can detect when a switch from the first garment 170 to the second garment 170 has occurred. In some embodiments, the usage trends tracked as shown in diagram 400 can be tracked and broken down for each individual garment 170 based on garment ID so that the number of hours and/or days of consecutive use of each garment can be logged. In such arrangements, a determination of the likelihood that a particular garment 170 has been used without being washed or changed can be made with a greater precision and/or accuracy than using usage trends without garment ID information. To further enhance the precision and/or accuracy of this determination, a specialized ID tag 190 can used with moisture or water detection as discussed below.

In one or more embodiments, garment ID tag or NFC tag 190 can include circuitry or some other mechanism that is capable of detecting when the ID tag 190 as come into contact with moisture or water. In such embodiments, a specialized ID tag or NFC tag 190 on garment 170 can include a built-in liquid sensor that is capable of detecting water coming into contact with the garment 170 or coming into contact with the garment receptacle 512 as shown in the embodiment of FIG. 5, which can indicate that the garment 170 likely has been washed. Such a liquid contact event can be detected by an appropriate NFC tag reader 510 and reported to the tracking device such as WCD remote patient data platform 316.

In the event that a liquid contact event has not been detected for an extended or predetermined period, a caution or warning can be provided to WCD system 10 and/or mobile communication device 110, or to another person or caregiver in order to address the situation if garment changes and/or washing are not observed for the extended or predetermined period. In such examples, the absence of detection of liquid contact with the ID tag 190 can indicate a higher likelihood that a particular garment 170 has not been washed than usage trends alone may indicate, although the scope of the disclosed subject matter is not limited in this respect. In some embodiments, information regarding the likelihood that the patient 182 has not changed or washed garment 170 over an extended or predetermined period can be provided to the patient's caregiver and/or physician to allow intervention before skin issues become a problem that may involve treatment or discontinuation of using the WCD system 10. Such a proactive approach to addressing potential skin irritation issues that may develop from extended wearing of garment 170 without changing or washing the garment 170 can result in an enhanced patient experience and increased patient compliance of using WCD system 10 which can save the lives of more patients 182.

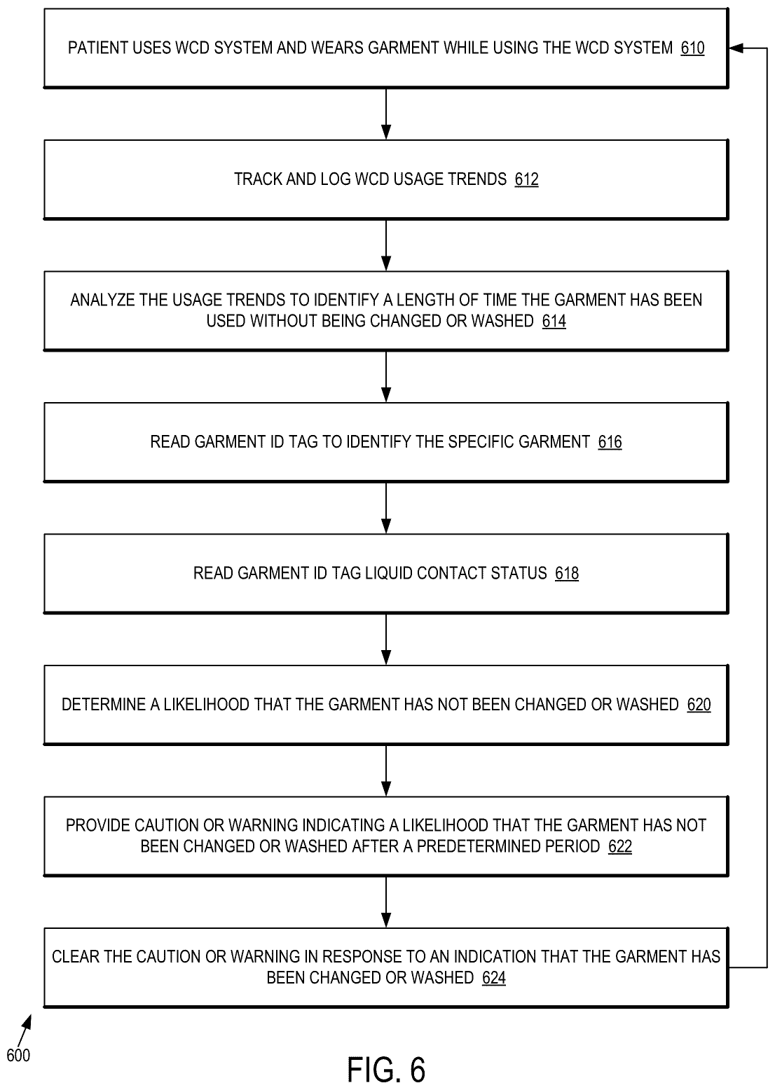

Referring now to FIG. 6, a flow diagram of a method to detect a likelihood that a garment of a WCD system has not been washed or changed in accordance with one or more embodiments will be discussed. Method 600 can be executed in whole or in part by any one or more of WCD system 10, mobile communication device 110, and/or WCD remote patient data platform 316. Furthermore, method 600 can include more or fewer operations than shown, in various orders other than shown in FIG. 6, and the scope of the disclosed subject matter is not limited in these respects. In addition, although liquid contact with the garment 170 is discussed for purposes of example, it should be noted that method 600 of FIG. 6 can be modified to include or substitute other ways to detect whether the garment 170 has been washed, for example using a temperature sensor to detect whether the garment 170 has been exposed to hot water from a washing machine, for example at a temperature of 130 degrees Fahrenheit or greater, or using an accelerometer to detect whether the garment 170 has been subject to agitation or spinning by a clothes washing machine or a clothes dryer. Method 600 can be modified accordingly, and the scope of the disclosed subject matter is not limited in these respects.

At operation 610, the patient 182 uses WCD system 10 and wears garment 170 while using WCD system 610. At operation 612, one or more usage trends can be tracked and logged, for example the usage trends as shown in and described with respect to FIG. 4. The usage trends can be analyzed at operation 614 to identify a length of time that garment 170 has been used by the patient 182 without being changed or washed. In some examples, such a determination at operation can be made based on the usage trends alone. In other examples, if garment includes an ID tag 190 comprising an NFC tag on which an ID or serial number of the garment 170 is stored and read by an appropriate NFC tag reader 510, the garment ID tag 190 can be read at operation 616 to identify the specific garment 170 being used with WCD system 10, for example as shown in and described with respect to FIG. 5. In further examples, in the event that garment ID tag 190 is configured to detect moisture, water, or liquid, the garment ID tag 190 can be read to determine a liquid contact status of the garment 170 at operation 618. In such examples, the ID tag 190 identifying information and/or liquid contact status information can be read and determined to assist in determining a status or likelihood that the garment 170 has been used and not changed or washed for an extended or predetermined period. In some examples, the ID tag 190 can include other sensors to detect wither or not the garment 170 has been washed. For example, the ID tag 190 can include a temperature sensor to detect whether the garment has been exposed to the hot water of a clothes washing machine. The hot water temperature of a clothes washing machine is likely to be 130 degrees Fahrenheit or greater which is greater than the temperature of the patient's body which is around 98 degrees Fahrenheit. In another example, the ID tag 190 can include an accelerometer to detect the agitation or spinning applied to the garment 170 by a clothes washing machine or a clothes drier. It is noted that these are merely examples of how the likelihood of the garment 170 having being washed can be detected, and the scope of the disclosed subject matter is not limited in this respect.

At operation 620, based at least in part on information obtained and analyzed at any one or more of operation 614, operation 616, and/or operation 618, a likelihood can be determined that the garment 170 has not been changed or washed for an extended or predetermined period. In the event it is likely that the garment 170 has not been changed or washed, a caution or warning can be provided at operation 622 indicating a likelihood that the garment 170 has not been changed or washed after an extended or predetermined period of use. Such an indication can be provided to the patient 182, for example via user interface 270 or via a user interface of mobile communication device 110. This indication also can be provided to a remote user via an interface of WCD remote patient data platform 316 and/or by transmitting a message or signal to the remote user to his or her own mobile communication device, although the scope of the disclosed subject matter is not limited in these respects. After an indication such as a caution or warning is provided at operation 622, or at any other appropriate time, the caution or warning can be cleared at operation 624 in response to an indication or detection that the garment 170 has been changed or washed. For example, if the patient 182 changes or washes the garment 170, the patient 182 can clear or reset the caution or warning using user interface 270 or a user interface of mobile communication device 110. In another example, once the WCD system 10 detects that the garment 170 has been changed to another fresh garment 170 by detecting the garment ID tag 190 information, the WCD system 10, mobile communication device 110, and/or WCD remote patient data platform can clear or reset the caution or warning. In yet another example, the caution or warning can be cleared or reset after a liquid contact event has been detected by garment ID tag 190 indicating that the garment 170 has been washed. Various other scenarios or combinations of data and/or events as discussed herein can be contemplated to result in the caution or warning being reset or cleared, and the scope of the disclosed subject matter is not limited in these respects. Once the caution or warning is cleared or reset, method 600 can continue at operation 610 with the washed or changed garment 170.

It should be noted that the WCD system 10 as described herein can be generalized to a patient monitoring system that can include action unit 180 with defibrillator 100 or without including defibrillator 100 in other embodiments. A patient monitoring system or a WCD system a described herein can be understood according to the following examples. In a first example, a patient monitoring system comprises a support structure comprising a garment configured to be worn by a patient, a plurality of patient parameter electrodes configured to contact the patient's skin when the patient is wearing the garment, a patient monitoring device configured to receive one or more patient parameters via the plurality of patient parameter electrodes when the patient is wearing the garment, a processor. The processor is configured to determine a likelihood whether the garment has been changed or washed after a predetermined period of use by the patient, and provide an indication regarding garment change or wash status based on the likelihood that the garment has been changed or washed. In a second example, the patient monitoring system further comprises a defibrillator system coupled with the patient monitoring device. The defibrillator comprises a plurality of defibrillator electrodes configured to be in electrical contact with the patient's skin when the patient is wearing the garment and when the defibrillator system is delivering therapy to the patient, and an energy storage system coupled with the plurality of defibrillator electrodes. In a third example, the processor is configured to determine whether to provide therapy to the patient based on the one or more patient parameters, and cause the energy storage system to deliver one or more therapeutic shocks to the patient responsive to the processor determining that therapy should be provided to the patient. In a fourth example, the processor is part of the defibrillator system. In a fifth example, the processor is part of the patient monitoring device.

In a sixth example, the processor is external to the patient monitoring device. In a seventh example, the processor is configured to track one or more usage trends of the patient monitoring system, and analyze the one or more usage trends to determine whether the garment has been changed or washed after a predetermined period of use by the patient. In an eight example, the patient monitoring system further comprises a garment identifier (ID) tag disposed in the garment to store garment identification information, and an ID tag reader to read the garment identification information from the garment ID tag. The processor is configured to determine whether the garment has been changed or washed after the predetermined period of use by the patient based at least in part on the garment identification information. In a ninth example, the garment ID tag comprises a liquid contact sensor and is configured to store liquid contact status information obtained by the liquid contact sensor, the ID tag reader is configured to read the liquid contact status information stored in the garment ID tag, and the processor is configured to determine whether the garment has been washed based at least in part on the liquid contact status information.

In a tenth example, the garment ID tag comprises a temperature sensor and is configured to store temperature information obtained by the temperature sensor, the ID tag reader is configured to read the temperature information stored in the garment ID tag, and the processor is configured to determine whether the garment has been washed based at least in part on the temperature information. In an eleventh example, the garment ID tag comprises an accelerometer and is configured to store acceleration information obtained by the accelerometer, the ID tag reader is configured to read the acceleration information stored in the garment ID tag, and the processor is configured to determine whether the garment has been washed based at least in part on the acceleration information. In a twelfth example, the patient monitoring system further comprises a communication module configured to transmit the one or more patient parameters to the processor. In a thirteenth example, the processor is configured to determine whether the garment has been changed or washed, and clear the indication regarding garment change or wash status in response to a user input, or in response the determination that the garment has been changed or washed.

In a fourteenth example, a remote patient data platform comprises a processor and a memory coupled with the processor, and a communication module. The processor is configured to receive information regarding one or more patient parameters of a patient monitoring system via the communication module, the patient monitoring system comprising a garment configured to be worn by a patient during use, determine a likelihood whether the garment has been changed or washed after a predetermined period of use by the patient, and provide an indication regarding garment change or wash status based on the likelihood whether the garment has been changed or washed. In a fifteenth example, the processor is configured to track one or more usage trends of the patient monitoring system based on the received information, and analyze the one or more usage trends to determine whether the garment has been changed or washed after a predetermined period of use by the patient. In a sixteenth example, the processor is configured to determine whether the garment has been changed or washed after a predetermined period of use by the patient based at least in part on garment identification information obtained from a garment identifier (ID) tag disposed in the garment to store garment identification information.

In a seventeenth example, a method to determine whether a garment has been changed or washed comprises tracking one or more usage trends of a patient's use of a patient monitoring system, analyzing the usage trends to identify a length of time the garment has been used without being changed or washed, determining a likelihood whether the garment has been changed or washed after a predetermined period of use by the patient, and providing an indication regarding garment change or wash status based on the likelihood that the garment has been changed or washed. In an eighteenth example, the method comprises determining the likelihood whether the garment has been changed or washed after a predetermined period of use by the patient is based at least in part on garment identification information obtained from a garment identifier (ID) tag disposed in the garment. In a nineteenth example, the method comprises determining the likelihood whether the garment has been washed is based at least in part on liquid contact status information, temperature information, acceleration information, or a combination thereof, obtained from a garment identifier (ID) tag disposed in the garment. In a twentieth example, the method comprises transmitting the garment change or wash status to a remote device coupled with the patient monitoring system.

Other embodiments include combinations and sub-combinations of features described or shown in the drawings herein, including for example, embodiments that are equivalent to: providing or applying a feature in a different order than in a described embodiment, extracting an individual feature from one embodiment and inserting such feature into another embodiment, removing one or more features from an embodiment, or both removing one or more features from an embodiment and adding one or more features extracted from one or more other embodiments, while providing the advantages of the features incorporated in such combinations and sub-combinations. As used in this paragraph, feature or features can refer to the structures and/or functions of an apparatus, article of manufacture or system, and/or the operations, acts, or modalities of a method.

Although the claimed subject matter has been described with a certain degree of particularity, it should be recognized that elements thereof can be altered by persons skilled in the art without departing from the spirit and/or scope of claimed subject matter. It is believed that the subject matter pertaining to power in a wearable cardioverter defibrillator (WCD) and many of its attendant utilities will be understood by the foregoing description, and it will be apparent that various changes can be made in the form, construction and/or arrangement of the components thereof without departing from the scope and/or spirit of the claimed subject matter or without sacrificing all of its material advantages, the form herein before described being merely an explanatory embodiment thereof, and/or further without providing substantial change thereto. It is the intention of the claims to encompass and/or include such changes.

What is claimed is:

1. A patient monitoring system, comprising:
a support structure comprising a garment configured to be worn by a patient;
a plurality of patient parameter electrodes configured to contact the patient's skin when the patient is wearing the garment;
a patient monitoring device configured to receive one or more patient parameters via the plurality of patient parameter electrodes when the patient is wearing the garment; and
a processor configured to:
determine a likelihood whether the garment has been changed or washed after a predetermined period of use by the patient, and
provide an indication regarding garment change or wash status based on the likelihood that the garment has been changed or washed.

2. The patient monitoring system of claim 1, further comprising a defibrillator system coupled with the patient monitoring device, the defibrillator comprising;
a plurality of defibrillator electrodes configured to be in electrical contact with the patient's skin when the patient is wearing the garment and when the defibrillator system is delivering therapy to the patient; and
an energy storage system coupled with the plurality of defibrillator electrodes.

3. The patient monitoring system of claim 2, wherein the processor is configured to:
determine whether to provide therapy to the patient based on the one or more patient parameters, and
cause the energy storage system to deliver one or more therapeutic shocks to the patient responsive to the processor determining that therapy should be provided to the patient.

4. The patient monitoring system of claim 2, wherein:
the processor is part of the defibrillator system.

5. The patient monitoring system of claim 1, wherein:
the processor is part of the patient monitoring device.

6. The patient monitoring system of claim 1, wherein:
the processor is external to the patient monitoring device.

7. The patient monitoring system of claim 1, wherein the processor is configured to:
track one or more usage trends of the patient monitoring system, and
analyze the one or more usage trends to determine whether the garment has been changed or washed after a predetermined period of use by the patient.

8. The patient monitoring system of claim 1, further comprising:
a garment identifier (ID) tag disposed in the garment to store garment identification information; and
an ID tag reader to read the garment identification information from the garment ID tag:
wherein the processor is configured to determine whether the garment has been changed or washed after the predetermined period of use by the patient based at least in part on the garment identification information.

9. The patient monitoring system of claim 8, wherein:
the garment ID tag comprises a liquid contact sensor and is configured to store liquid contact status information obtained by the liquid contact sensor;
the ID tag reader is configured to read the liquid contact status information stored in the garment ID tag; and
the processor is configured to determine whether the garment has been washed based at least in part on the liquid contact status information.

10. The patient monitoring system of claim 8, wherein:
the garment ID tag comprises a temperature sensor and is configured to store temperature information obtained by the temperature sensor;
the ID tag reader is configured to read the temperature information stored in the garment ID tag; and
the processor is configured to determine whether the garment has been washed based at least in part on the temperature information.

11. The patient monitoring system of claim 8, wherein:
the garment ID tag comprises an accelerometer and is configured to store acceleration information obtained by the accelerometer;
the ID tag reader is configured to read the acceleration information stored in the garment ID tag; and
the processor is configured to determine whether the garment has been washed based at least in part on the acceleration information.

12. The patient monitoring system of claim 1, further comprising:
a communication module configured to transmit the one or more patient parameters to the processor.

13. The patient monitoring system of claim 1, wherein the processor is configured to:
determine whether the garment has been changed or washed; and
clear the indication regarding garment change or wash status in response to a user input, or in response the determination that the garment has been changed or washed.

14. A remote patient data platform, comprising:
a processor and a memory coupled with the processor; and
a communication module;
wherein the processor is configured to:
receive information regarding one or more patient parameters of a patient monitoring system via the communication module, the patient monitoring system comprising a garment configured to be worn by a patient during use;
determine a likelihood whether the garment has been changed or washed after a predetermined period of use by the patient; and
provide an indication regarding garment change or wash status based on the likelihood whether the garment has been changed or washed.

15. The remote patient data platform of claim 14, wherein the processor is configured to:
track one or more usage trends of the patient monitoring system based on the received information; and
analyze the one or more usage trends to determine whether the garment has been changed or washed after a predetermined period of use by the patient.

16. The remote patient data platform of claim 14, wherein the processor is configured to:
determine whether the garment has been changed or washed after a predetermined period of use by the patient based at least in part on garment identification information obtained from a garment identifier (ID) tag disposed in the garment to store garment identification information.

17. A method to determine whether a garment has been changed or washed, the method comprising:
tracking one or more usage trends of a patient's use of a patient monitoring system;
analyzing the usage trends to identify a length of time the garment has been used without being changed or washed;
determining a likelihood whether the garment has been changed or washed after a predetermined period of use by the patient; and
providing an indication regarding garment change or wash status based on the likelihood that the garment has been changed or washed.

18. The method of claim 17, wherein:
determining the likelihood whether the garment has been changed or washed after a predetermined period of use by the patient is based at least in part on garment identification information obtained from a garment identifier (ID) tag disposed in the garment.

19. The method of claim 17, wherein:
determining the likelihood whether the garment has been washed is based at least in part on liquid contact status information, temperature information, acceleration information, or a combination thereof, obtained from a garment identifier (ID) tag disposed in the garment.

20. The method of claim 17, further comprising:
transmitting the garment change or wash status to a remote device coupled with the patient monitoring system.
